(19) United States
(12) Patent Application Publication
(10) Pub. No.: US 2026/0355393 A1
(43) Pub. Date: Nov. 12, 2026

(54) MULTILAYERED BIODEGRADABLE DRUG DELIVERY DEVICE

(71) Applicant: Sriyan Daggubati, Danville, CA (US)

(72) Inventor: Sriyan Daggubati, Danville, CA (US)

(21) Appl. No.: 19/671,326

(22) Filed: May 8, 2026

Related U.S. Application Data
(60) Provisional application No. 63/802,623, filed on May 9, 2025.

(51) Int. Cl. A61L 31/10; A61L 31/04; A61L 31/16

(52) U.S. Cl. CPC A61L 31/10; A61L 31/041; A61L 31/045; A61L 31/16; A61L 2300/22; A61L 2300/602; A61L 2300/606; A61L 2420/02; A61L 2420/06; A61L 2420/08; A61L 2430/30

(57) ABSTRACT

A multilayered biodegradable drug delivery device is disclosed. The multilayered biodegradable drug delivery device comprises a first layer, a second layer, and an intermediate layer disposed between the first layer and the second layer. The first layer comprises 85% PLGA and 15% gelatin, the intermediate layer comprises 75% PLGA and 25% gelatin, and the second layer comprises 50% PLGA and 50% gelatin. Further, the multilayered biodegradable drug delivery device comprises a specialized pro-resolving mediator (SPM) having a Resolvin D1 (RvD1) solution. The SPM is loaded between the first layer and the intermediate layer, and between the intermediate layer and the second layer. The progressive gradient of PLGA and gelatin content across the first layer, the intermediate layer, and the second layer provides sustained release of the RvD1 solution to targeted vascular injury sites. The released RvD1 inhibits nuclear factor-κB activation to reduce inflammation, vascular smooth muscle cell proliferation and migration.

### TECHNICAL FIELD

The subject matter of the proposed technology relates to devices for drug delivery, methods of making the devices, and methods of using the devices for drug delivery. More particularly, the subject matter pertains to multilayered biodegradable thin films designed for the controlled release of resolvins, particularly Resolvin D1 (RvD1), at vascular injury sites to facilitate localized inflammation resolution and tissue regeneration.

### BACKGROUND

Vascular injuries and diseases often lead to persistent inflammation, hindering natural healing and increasing the risk of complications like graft failure and thrombosis. Specialized pro-resolving mediators (SPMs), including Resolvin D1 (RvD1), derived from omega-3 fatty acids, offer a promising therapeutic avenue by actively promoting the resolution of inflammation and tissue repair. RvD1, in particular, exhibits potent anti-inflammatory and pro-resolving properties by modulating immune cell activity and enhancing the clearance of cellular debris.

Current systemic delivery methods for specialized pro-resolving mediators (SPMs), such as intravenous, subcutaneous, or intraperitoneal administration, are limited by poor tissue specificity and low bioavailability. These approaches often result in widespread distribution of the therapeutic agent, thereby reducing its efficacy at localized sites of vascular injury. Additionally, the systemic delivery methods often result in poor targeting and limited bioavailability. Further, the clinical translation of RvD1 is hampered by its rapid degradation and the difficulty in achieving sustained, localized delivery to the injured vasculature, limiting its therapeutic efficacy.

However, existing delivery systems for delivering SPMs often lead to rapid diffusion away from the target inflammation site, resulting in insufficient local drug concentration and reduced therapeutic efficacy. Further, the RvD1 has a short biological half-life, necessitating repeated administration to maintain effective levels. Conventional delivery systems further lack directional release toward the vessel wall, leading to inefficient targeting of the affected vascular tissue.

Therefore, there is a need for advanced drug delivery devices capable of delivering RvD1 directly to injured vascular tissue in a sustained and controlled manner, thereby enhancing therapeutic efficacy while minimizing off-target effects on surrounding healthy tissues.

### SUMMARY

The proposed technology discloses a multilayered biodegradable drug delivery device. The multilayered biodegradable drug delivery device comprises a first layer, a second layer, and an intermediate layer disposed between the first layer and the second layer. The first layer comprises about 85% PLGA and about 15% gelatin, the intermediate layer comprises about 75% PLGA and about 25% gelatin, and the second layer comprises about 50% PLGA and about 50% gelatin. Further, the multilayered biodegradable drug delivery device comprises a specialized pro-resolving mediator (SPM). The SPM is loaded between the first layer and the intermediate layer, and between the intermediate layer and the second layer. In an embodiment, the SPM is Resolvin. The Resolvin includes Resolvin D1 (RvD1). In an embodiment, the RvD1 is dissolved in ethanol to obtain a Resolvin D1 (RvD1) solution that is loaded between the first layer and the intermediate layer, and between the intermediate layer and the second layer. The progressive gradient of PLGA and gelatin content across the first layer, the intermediate layer, and the second layer provides directional and sustained release of the SPM such as a Resolvin D1 (RvD1) solution, to a targeted vascular inflammation site.

In an embodiment, a method of treating a vascular condition using a multilayered biodegradable drug delivery device. The method comprises applying the multilayered biodegradable drug delivery device to a blood vessel of a subject at a targeted site. The device comprises the first layer comprising about 85% PLGA and about 15% gelatin, the intermediate layer comprising about 75% PLGA and about 25% gelatin, and the second layer comprising about 50% PLGA and about 50% gelatin. The device comprises the specialized pro-resolving mediator (SPM) loaded between the first layer and the intermediate layer and between the intermediate layer and the second layer. In an embodiment, the subject is a human. In an embodiment, the first layer is oriented toward an outer surface of the blood vessel and the second layer is oriented toward an inner surface of the blood vessel.

The method further comprises facilitating a localized release of the RvD1 at the targeted site. In an embodiment, the targeted site is a site of a vascular condition selected from the group consisting of vascular injury, vascular inflammation, ischemia-reperfusion injury, endotoxin-induced vascular damage, vascular trauma, graft failure, and thrombosis. In an embodiment, the released RvD1 in the subject inhibits a nuclear factor-κB (NF-κB) activation in vascular smooth muscle cells. Further, the device reduces vascular smooth muscle cell proliferation and vascular smooth muscle cell migration in the subject upon releasing the RvD1.

### BRIEF DESCRIPTION OF DRAWINGS

Exemplary embodiments of the proposed technology will be understood and appreciated fully from the following detailed description read in conjunction with the drawings in which,

FIG. 1 illustrates a drug delivery device 100 of the proposed technology, in accordance with an embodiment; and

FIG. 2A is a graph illustrating cumulative drug release in SFM media and PBS media, in accordance with an embodiment;

FIG. 2B is a graph illustrating drug stability in ethanol, a phosphate-buffered saline (PBS) media and a serum-free media (SFM) media, in accordance with an embodiment;

FIG. 3A is a graph illustrating a cumulative drug release from the drug delivery device 100 in a homogenized state and an intact state in ethanol, in accordance with an embodiment;

FIG. 3B is a graph illustrating the cumulative drug release from the drug delivery device 100 in homogenized state in PBS, in accordance with an embodiment;

FIG. 4A is a graph illustrating a directional release of a RvD1 drug using a bi-layered device, in accordance with an embodiment;

FIG. 4B is a graph illustrating the directional release of the RvD1 drug using the drug delivery device 100, in accordance with an embodiment;

FIG. 5A shows the effect of the RvD1 drug-loaded multilayered biodegradable drug delivery device on TNF-α-induced NF-κB activation in vascular smooth muscle cells, in accordance with an embodiment;

FIG. 5B shows the effect of the RvD1-loaded multilayered biodegradable drug delivery device on vascular smooth muscle cell proliferation, in accordance with an embodiment;

FIG. 5C shows the effect of the RvD1-loaded multilayered biodegradable drug delivery device on vascular smooth muscle cell migration, in accordance with an embodiment;

FIG. 6 is a graph illustrating the drug delivered by the drug delivery device 100 to an inner surface of a blood vessel and an outer surface of the blood vessel, in an ex vivo system, in accordance with an embodiment; and

FIG. 7 is a graph illustrating the drug delivered by the drug delivery device 100 to a left carotid artery and a right carotid artery, in an in-vivo system, in accordance with an embodiment.

### DETAILED DESCRIPTION

The following detailed description includes references to the accompanying drawings, which form part of the detailed description. The drawings show illustrations in accordance with example embodiments. The numerals in the figure represent like elements throughout the several views, exemplary embodiments of the present disclosure are described. For convenience, only some elements of the same group may be labelled with numerals. The purpose of the drawings is to describe exemplary embodiments and not for production. Therefore, features shown in the figures are chosen for convenience and clarity of presentation only. Moreover, the language used in this disclosure has been principally selected for readability and instructional purposes and may not have been selected to delineate or circumscribe the inventive subject matter, resort to the claims being necessary to determine such inventive subject matter. These example embodiments are described in enough detail to enable those skilled in the art to practice the present subject matter. However, it will be apparent to a person of ordinary skill in the art that the proposed technology may be practiced without these specific details. The embodiments can be combined, other embodiments can be utilized, or structural and logical changes can be made without departing from the scope of the invention. The following detailed description is, therefore, not to be taken in a limiting sense.

In this document, the terms “a” or “an” are used, as is common in patent documents, to include one or more than one. In this document, the term “or” is used to refer to a non-exclusive “or” such that “a or b” includes “a but not b,” “b but not a,” and “a and b,” unless otherwise indicated.

Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

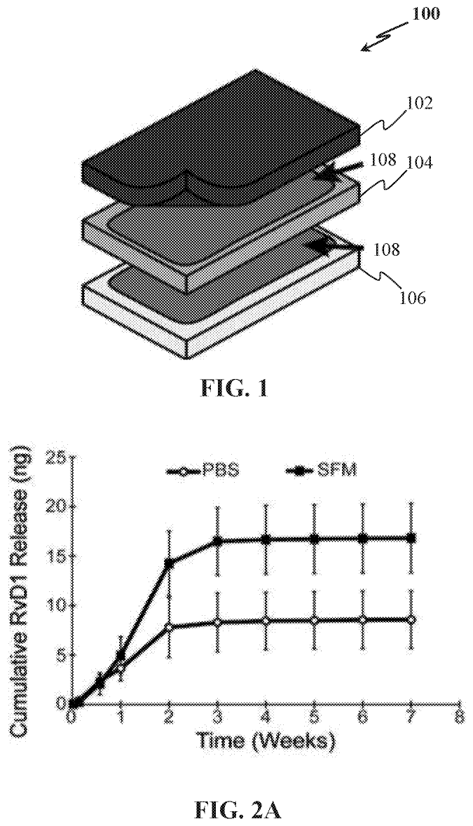

Referring to FIG. 1, a multilayered biodegradable drug delivery device 100 is disclosed. The multilayered biodegradable drug delivery device 100 of the proposed technology is a therapeutic film which is biodegradable and gelatin based (referred to as biodegradable film hereafter in the document). The multilayered biodegradable drug delivery device 100 is designed for the controlled and sustained release of RvD1. The multilayered biodegradable drug delivery device 100 is referred to as drug delivery device 100 throughout the specification.

In an embodiment, the disclosed approach enables targeted and sustained delivery of Resolvin D1 (RvD1) directly to sites of vascular injury. This localized administration maximizes the therapeutic efficacy of the RvD1 at the site of damage while substantially reducing systemic exposure and limiting potential effects on adjacent healthy vascular tissue.

In an embodiment, the drug delivery device 100 is a multilayered biodegradable film.

In an embodiment, the drug delivery device 100 is tri-layered structure.

In an embodiment, each layer of the drug delivery device 100 comprises poly(lactic-co-glycolic acid) (PLGA) films. In one embodiment, the PLGA films are coated with gelatin solution and referred to as gelatin-coated PLGA film. In an embodiment, the gelatin coating has a thickness of about 1 μm to about 100 μm on each PLGA film. In another embodiment, the gelatin coating has a thickness of about 5 μm to about 50 μm on each PLGA film. In another embodiment, the gelatin coating has a thickness of about 5 μm to about 50 μm on each PLGA film. In a preferred embodiment, the gelatin coating has a thickness of about 10 μm on each PLGA film.

In an embodiment, the drug delivery device 100 comprises a first layer 102, an intermediate layer 104 and a second layer 106. The intermediate layer 104 is disposed between the first layer 102 and the second layer 106.

In an embodiment, the first layer 102 comprises about 70% to about 95% poly(lactic-co-glycolic acid) (PLGA) and about 5% to about 30% gelatin. In another embodiment, the first layer 102 comprises about 80% to about 90% PLGA and about 10% to about 20% gelatin.

In an embodiment, the intermediate layer 104 comprises about 55% to about 85% PLGA and about 15% to about 45% gelatin. In another embodiment, the intermediate layer 104 is about 65% to about 80% PLGA and about 20% to about 35% gelatin.

In an embodiment, the second layer 106 comprises about 25% to about 70% PLGA and about 30% to about 75% gelatin. In another embodiment, the second layer 106 is about 40% to about 60% PLGA and about 40% to about 60% gelatin.

In the preferred embodiment, the first layer 102 is composed of 85% poly(lactic-co-glycolic acid) (PLGA) and 15% gelatin, the intermediate layer 104 is composed of 75% PLGA and 25% gelatin, the second layer 106 is composed of 50% PLGA and 50% gelatin.

Further, the drug delivery device 100 comprises a specialized pro-resolving mediator (SPM). The SPM is loaded between the first layer 102 and the intermediate layer 104, and between the intermediate layer 104 and the second layer 106. In an embodiment, the SPM is Resolvin. The Resolvin includes Resolvin D1 (RvD1). In an embodiment, the RvD1 is dissolved in a pharmaceutically acceptable solvent to form an RvD1 solution. In an embodiment, the RvD1 solution has a concentration in the range of about 0.1 μg/mL to about 1,000 μg/mL. In another embodiment, the RvD1 solution has a concentration in the range of about 1 μg/mL to about 250 μg/mL. In another embodiment, the RvD1 solution has a concentration is in the range of about 10 μg/mL to about 100μg/mL. In the preferred embodiment, the RvD1 solution has a concentration of about 100 μg/mL.

In an embodiment, the amount of RvD1 loaded between the first layer and the intermediate layer and between the intermediate layer and the second layer is in the range of about 1 ng to about 10 μg. In another embodiment, the amount of RvD1 loaded between the first layer and the intermediate layer and between the intermediate layer and the second layer is in the range of about 1 ng to about 1 μg. In another embodiment, the amount of RvD1 loaded between the first layer and the intermediate layer and between the intermediate layer and the second layer is in the range of about 10 ng to about 500 ng. In the preferred embodiment, the amount of RvD1 loaded between the first layer and the intermediate layer and between the intermediate layer and the second layer is about 100 ng.

In an embodiment, the RvD1 is dissolved in a pharmaceutically acceptable solvent to obtain a Resolvin D1 (RvD1) solution 108 that is loaded between the first layer 102 and the intermediate layer 104, and between the intermediate layer 104 and the second layer 106. The pharmaceutically acceptable solvent might include, but not limited to, ethanol, methanol, isopropanol, dimethyl sulfoxide, acetonitrile, polyethylene glycol, propylene glycol, glycerol, phosphate-buffered saline containing a solubilizing agent, an albumin-containing solution, a cyclodextrin-containing solution, a lipid-based carrier, or combinations thereof. In a preferred embodiment, the RvD1 is dissolved in ethanol.

The progressive gradient of PLGA and gelatin content across the first layer 102, the intermediate layer 104, and the second layer 106 provides directional and sustained release of the SPM, such as a Resolvin D1 (RvD1) solution 108, to a targeted vascular inflammation site.

In an embodiment, the targeted site is a site of a vascular condition selected from the group consisting of vascular injury, vascular inflammation, ischemia-reperfusion injury, endotoxin-induced vascular damage, vascular trauma, graft failure, and thrombosis. In an embodiment, the released RvD1 in the subject inhibits a nuclear factor-κB (NF-κB) activation in vascular smooth muscle cells. They suppress the NF-κB signalling pathway, which regulates immune responses and reduces inflammation. Further, the device reduces vascular smooth muscle cell proliferation and vascular smooth muscle cell migration in the subject upon releasing the RvD1.

In an embodiment, the RvD1 is loaded between the first layer 102 and the intermediate layer 104, and between the intermediate layer 104 and the second layer 106. In an embodiment, the RvD1 loaded is in the range of about 1 ng to about 10 μg. In another embodiment, the RvD1 loaded is in the range of about 10 ng to about 1 μg. In the preferred embodiment, the amount of RvD1 loaded might be about 100 ng. The preferred RvD1 concentration and loading amount might be adjusted depending on the desired release profile, therapeutic effect, and coating characteristics of the interface.

In an embodiment, 100 ng RvD1 solution 108 is loaded in between the layers of the drug delivery device 100. In other words, 100 ng RvD1 solution 108 is loaded in between the first layer 102 and the intermediate layer 104. Similarly, 100 ng RvD1 solution 108 is loaded between the intermediate layer 104 and the second layer 106.

In an embodiment, the thickness of the drug delivery device 100 of the proposed technology is less than 100 μm. In an embodiment, the drug delivery device 100 comprises the first layer with a thickness in the range of 22-30 μm, an intermediate layer with a thickness in the range of 8.8-10.1 μm, and a second layer with a thickness in the range of 11.27-12.73 μm. In the preferred embodiment, the thickness of the first layer ranges from 26 μm, the thickness of the intermediate layer is 9.9 μm, and the second layer is 12 μm.

### Experimental Results

### In Vitro Release in Different Elution Buffers

The release kinetics of Resolvin D1 from the drug delivery device 100 designed for targeted drug release were examined. Long-term release studies revealed a biphasic release profile, with a quicker and higher cumulative drug release observed in serum-free media (SFM) compared to phosphate-buffered saline (PBS). The biphasic release profile means the drug is released in two distinct stages over time, including an initial phase and a secondary phase. In initial phase, a relatively rapid release of RvD1 drug occurs early, typically due to the RvD1 drug located near or on the surface of the device. This provides an immediate therapeutic effect at the target site. In the secondary phase, the RvD1 drug is released more slowly and controlled as it diffuses through the tri-layered structure having PLGA and gelatin, thereby maintaining therapeutic levels over an extended period.

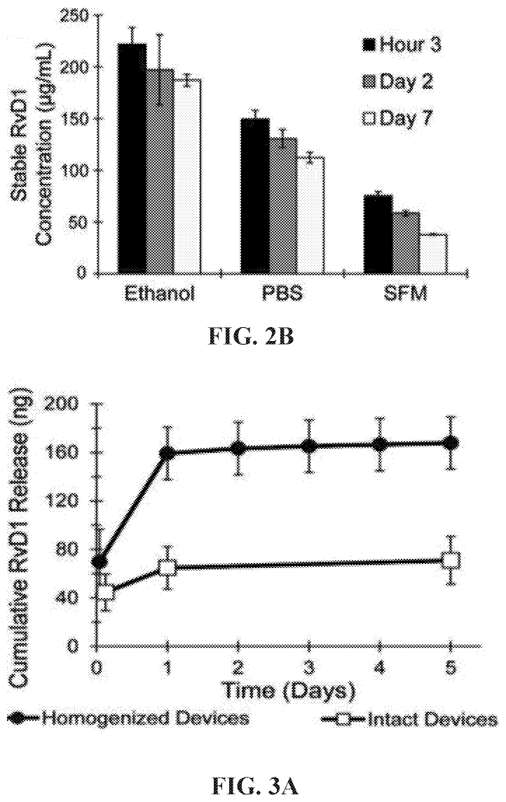

Referring to FIG. 2A, after one week of elution, the drug delivery device 100 in SFM released 4.96 (±1.89) ng of Resolvin D1, while those in PBS released 3.67 (±1.26) ng. At three weeks, the total cumulative release was 16.5 (±3.41) ng in SFM and 8.29 (±2.96) ng in PBS. The additional release between weeks 3 and 7 was 0.283 ng for PBS and 0.345 ng for SFM.

Although Resolvin D1 showed some degradation in the elution buffers, its stability was greater in PBS than in SFM. Nevertheless, the cumulative release in SFM was nearly double that in PBS, indicating that the drug delivery device 100 degradation may have enhanced drug release in SFM (refer: FIG. 2A).

### Drug Stability

To evaluate the stability of RvD1 in various media, samples were prepared by adding 1 μL of stock RvD1 (100 μg/mL) to 0.5 mL of SFM, PBS, and ethanol. Triplicate samples for each condition at 37 °C for 2 and 7 days were incubated, with one set being analyzed right after preparation. This method enabled the assessment of any degradation of the RvD1 over time in each medium. FIG. 2B showed that the stability of the drug RvD1 is better in ethanol compared to PBS and SFM. The drug RvD1 showed poor stability in SFM.

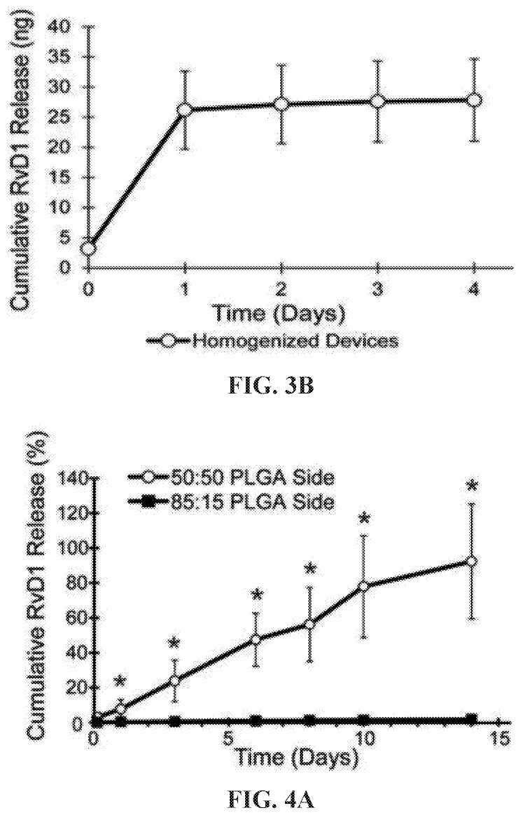

Further, to analyze drug stability, homogenization was performed to assess the total loading of the RvD1 in the drug delivery device 100 which was homogenised (homogenized) and the drug delivery device 100 in intact form, followed by elution using either PBS or ethanol. Referring to FIG. 3A, the total amount of the RvD1 recovered from the homogenized state of the drug delivery device 100 eluted in ethanol was 168 (±21.5) ng, which accounted for 83.9% of the loaded drug (Lee & Lim, 2020). In contrast, intact state of the drug delivery device 100 eluting in ethanol yielded 71.6 (±19.7) ng, or 35.8% of the loaded drug. Referring to FIG. 3B, the drug delivery device 100 when homogenised with PBS as the elution buffer resulted in a recovery of only 27.8 (±3.41) ng, or 13.9% of the drug-loaded. These findings suggest that surface area, enhanced by degradation, plays a crucial role in the elution of the RvD1 drug.

### Directional Release Studies

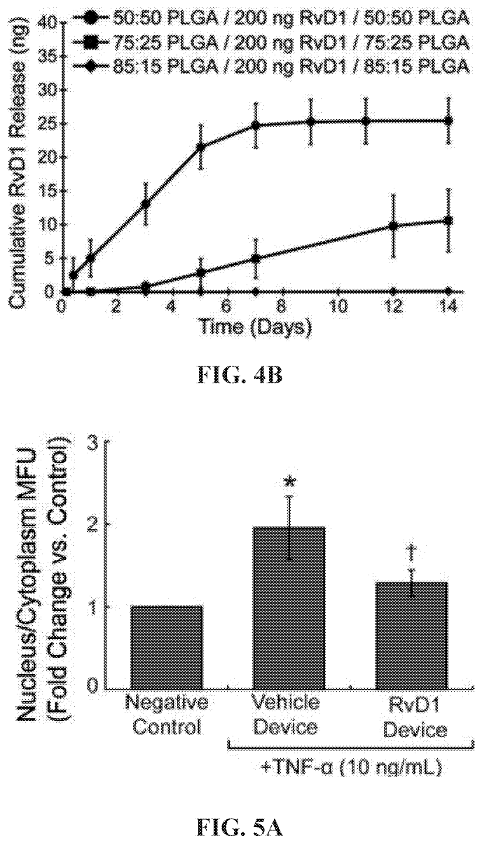

The directional release from the drug delivery device 100 was evaluated using a two-sided diffusion chamber, which allowed for the isolation of the SFM exposed to each side of the devices (Frerichs & Wendel, 2021). Statistically significant differences in release were noted as early as day 1, with 97.9% of the total cumulative amount of RvD1 released from the 50:50 gelatin side by day 14. Referring to FIG. 4A, a bi-layered structure having at least one layer with 50 PLGA: 50 gelatin and another layer with 85 PLGA: 15 gelatin that is compared with the drug device of the proposed technology. In FIG. 4B, the drug delivery device 100 showed directional RvD1 release with significant differences between sides (p<0.05), suggesting better control compared to bi-layered devices (FIG. 4A). The drug delivery device 100 exhibits distinct directional release properties, with a notably faster release observed from the 50 PLGA: 50 gelatin i.e. second layer 106 and significantly lower release from the 85 PLGA: 15 gelatin side i.e. the first layer 102. The directional release is preferentially oriented toward the targeted site from the drug delivery device 100.

### In Vitro Biological Assays

Primary cell cultures of endothelial cells (ECs) and vascular smooth muscle cells (VSMCs) were obtained from human greater saphenous veins that were discarded during bypass surgeries for biological evaluation (Elzoghby & Elgindy, 2020). The drug delivery device 100 containing Resolvin D1 (RvD1) or an ethanol control were positioned in transwell inserts above the cells to investigate the biological effects of RvD1 (Elzoghby & Elgindy, 2020). For the nuclear factor-κB (NF-κB) assay, VSMCs were treated with the drug delivery device 100 for 18 hours and then stimulated with TNF-α. After treatment, the cells were fixed and stained with a fluorescent dye to visualize the nuclear translocation of NF-κB using a fluorescent microscope (Machein & Plate, 2019). In the cell proliferation assay, VSMCs were serum-starved and stimulated with media. After exposure to the drug delivery device 100, cell counts were conducted to evaluate the impact of RvD1 on proliferation (Elzoghby & Elgindy, 2020). Migration assays involved creating a wound in a confluent VSMC monolayer, with the drug delivery device 100 loaded with RvD1 solution were placed in the wells to assess changes in cell migration over 24 hours; photographs of the wounds were taken at regular intervals to quantify migration (Machein & Plate, 2019). Lastly, cells were exposed to a vehicle device (which is conventionally known) and the drug delivery device 100 of the claimed proposed technology) to evaluate cytotoxicity, and cell viability was measured using an MTT assay, which quantifies viable cells based on their metabolic activity.

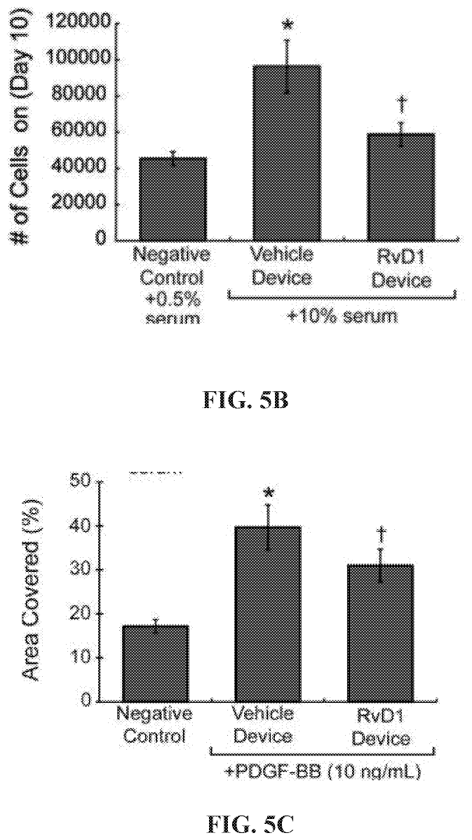

The vehicle device is a control device having substantially the same layers as the multilayered as the RvD1-loaded drug delivery device, but without RvD1. In one embodiment, the vehicle device comprises the same first layer, intermediate layer, and second layer configuration and is treated with the same solvent or carrier used for RvD1 loading, such as ethanol, without inclusion of RvD1. The vehicle device controls for the effects of the polymeric layers, gelatin content, device structure, and loading solvent, whereas the multilayered biodegradable drug delivery device comprises RvD1 loaded between adjacent layers for localized and sustained release to a targeted vascular site. The FIGS. 5A to 5B are the experimental results to illustrate the biological activity of RvD1 released from the drug delivery device 100 using vascular smooth muscle cells. The experiments involve vascular smooth muscle cells exposed to the RvD1-loaded multilayered biodegradable drug delivery device, cells exposed to the vehicle device, and the negative control cells. The vehicle device had the same multilayer PLGA/gelatin configuration as the RvD1-loaded device, but did not contain active RvD1. Instead, the vehicle device contained the solvent or carrier used for loading RvD1, such as ethanol, without RvD1. The negative control refers to cells maintained without exposure to the RvD1-loaded device. In the nuclear factor-κB (NF-κB) assay, the negative control may include cells not stimulated with TNF-α. The TNF-α might be Tumor Necrosis Factor-alpha (TNF-α).

Referring to FIG. 5A, the effect of the drug delivery device 100 with loaded RvD1 solution on TNF-α-induced NF-κB activation in vascular smooth muscle cells is evaluated. NF-κB activation was assessed by measuring nuclear translocation of the p65 subunit. Compared with the vehicle device, the drug delivery device 100 with loaded RvD1 solution reduced nuclear p 65 translocation by about 36%, indicating that RvD1 released from the device inhibited inflammatory nuclear factor-κB (NF-κB) signalling.

Referring to FIG. 5B, the effect of the drug delivery device 100 with loaded RvD1 solution on vascular smooth muscle cell proliferation is evaluated. Cells exposed to the RvD1-loaded device showed about a 39% reduction in proliferation compared with the vehicle device, indicating that RvD1 released from the drug delivery device 100 with loaded RvD1 solution reduced vascular smooth muscle cell growth.

Referring to FIG. 5C, the effect of the drug delivery device 100 with loaded RvD1 solution on vascular smooth muscle cell migration is evaluated. In the migration assay, a wound or gap was created in a confluent vascular smooth muscle cell monolayer, and the extent of wound closure was monitored over time. Cells exposed to the drug delivery device 100 with loaded RvD1 solution showed about a 22% reduction in migration compared with the vehicle device, indicating that RvD1 released from the device reduced vascular smooth muscle cell motility.

FIGS. 5A, 5B, and 5C demonstrate that the drug delivery device 100 with loaded RvD1 solution retains biological activity after loading and release. The released RvD1 suppresses inflammatory signalling, reduces vascular smooth muscle cell proliferation, and reduces vascular smooth muscle cell migration, supporting the use of the drug delivery device 100 for localized treatment of vascular inflammation, vascular injury, graft failure, restenosis, thrombosis, and related vascular conditions.

### Ex Vivo and in Vivo Delivery

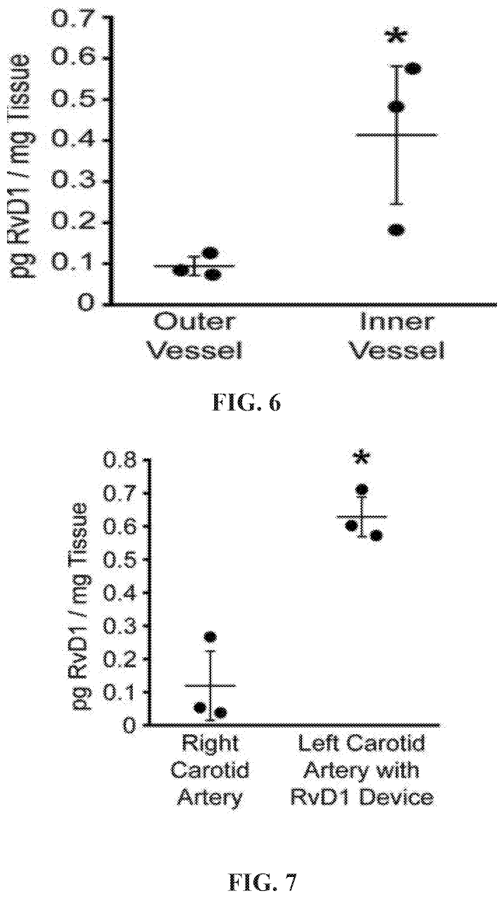

A validated method was used to extract Resolvin D1 (RvD1) from rabbit aortic tissue to study short-term unidirectional release in an ex vivo pulsatile flow system (Frerichs & Wendel, 2021). After 24 hours, an inner surface of the blood vessels had higher RvD1 levels (0.41 ±0.17 pg/mg) vs. an outer surface of the blood vessels (0.094 ±0.023 pg/mg) as shown in FIG. 6. When the tri-layered form of the drug delivery device 100 is surgically implanted at the targeted site, the first layer 102 is positioned toward the outer surface of the blood vessels and the second layer 106 is positioned toward the inner surface of the blood vessels.

Referring to FIG. 7, in vivo studies demonstrated that applying the drug delivery device 100 around the left carotid artery led to significantly higher levels of immunoreactive Resolvin D1 compared to untreated controls in the opposite artery one hour after application, with levels recorded at 0.63 (±0.060) pg per mg of tissue versus 0.12 (±0.10) pg per mg in the untreated artery (Chen & Xu, 2022). These results highlight the ability of the gelatin-based devices for targeted and sustained drug delivery in vascular applications.

The drug delivery device 100 of the proposed technology is capable of delivering therapeutically effective concentrations of Resolvin D1 (RvD1) to vascular tissues in both ex vivo and in vivo experimental models. Its flexible and conformable structure enables direct attachment to blood vessels, rendering it suitable for the treatment of various acute and sub-acute inflammatory conditions, including ischemia-reperfusion injury, endotoxin-induced vascular damage, and other forms of vascular trauma. This drug delivery device 100 offers considerable therapeutic promise for a broad spectrum of surgical procedures, where localized, sustained release of pro-resolving lipid mediators such as RvD1 may promote tissue repair and inflammation resolution.

The processes described above is described as a sequence of steps, this was done solely for the sake of illustration. Accordingly, it is contemplated that some steps may be added, some steps may be omitted, the order of the steps may be re-arranged, or some steps may be performed simultaneously.

Although embodiments have been described with reference to specific example embodiments, it will be evident that various modifications and changes may be made to these embodiments without departing from the broader spirit and scope of the system and process or method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

What is claimed is:

1. A multilayered biodegradable drug delivery device comprising:
a first layer comprising poly(lactic-co-glycolic acid) (PLGA) and gelatin;
a second layer comprising PLGA and gelatin;
an intermediate layer comprising PLGA and gelatin, wherein the intermediate layer is disposed between the first layer and the second layer; and
a specialized pro-resolving mediator (SPM) loaded between the first layer and the intermediate layer, and between the intermediate layer and the second layer,
wherein the first layer, the intermediate layer, and the second layer comprise differing relative amounts of PLGA and gelatin to provide a directional release of the SPM to a targeted site from the drug delivery device.

2. The multilayered biodegradable drug delivery device of claim 1, wherein:
the first layer comprises about 70% to about 95% of PLGA and about 5% to about 30% of gelatin;
the intermediate layer comprises about 55% to about 85% of PLGA and about 15% to about 45% of gelatin; and
the second layer comprises about 25% to about 70% of PLGA and about 30% to about 75% of gelatin.

3. The multilayered biodegradable drug delivery device of claim 2, wherein the first layer comprises about 85% PLGA and about 15% gelatin; the intermediate layer comprises about 75% PLGA and about 25% gelatin, and the second layer comprises about 50% PLGA and about 50% gelatin.

4. The multilayered biodegradable drug delivery device of claim 1, wherein the SPM is a Resolvin D1 (RvD1).

5. The multilayered biodegradable drug delivery device of claim 4, wherein the Resolvin D1 (RvD1) is dissolved in a pharmaceutically acceptable solvent to obtain a Resolvin D1 (RvD1) solution for loading between the first layer and the intermediate layer and between the intermediate layer and the second layer, wherein the pharmaceutically acceptable solvent comprises any one of ethanol, methanol, isopropanol, dimethyl sulfoxide, acetonitrile, polyethylene glycol, propylene glycol, glycerol, phosphate-buffered saline containing a solubilizing agent, an albumin-containing solution, a cyclodextrin-containing solution, a lipid-based carrier, or combinations thereof.

6. The multilayered biodegradable drug delivery device of claim 5, wherein the RvD1 solution loaded between the first layer and the intermediate layer and between the intermediate layer and the second layer, is in the range of about 1 ng to about 10 μg.

7. The multilayered biodegradable drug delivery device of claim 6, wherein about 100 ng of RvD1 solution is loaded between the first layer and the intermediate layer, and about 100 ng of RvD 1 solution is loaded between the intermediate layer and the second layer.

8. The multilayered biodegradable drug delivery device of claim 1, wherein the relative gelatin content increases progressively from the first layer to the second layer, wherein the second layer has a higher gelatin content compared to the intermediate layer, and the intermediate layer has a higher gelatin content compared to the first layer.

9. The multilayered biodegradable drug delivery device of claim 1, wherein the device has a total thickness of less than 100 μm.

10. The multilayered biodegradable drug delivery device of claim 9, wherein:
the first layer has a thickness in the range of 22-30 μm;
the intermediate layer has a thickness in the range of 8.8-10.1 μm; and
the second layer has a thickness in the range of 11.27-12.73 μm.

11. The multilayered biodegradable drug delivery device of claim 10, wherein the thickness of the first layer is 26 μm, the thickness of the intermediate layer is 9.9 μm, and the thickness of the second layer is 12 μm.

12. The multilayered biodegradable drug delivery device of claim 1, configured to provide a biphasic release profile of the SPM.

13. The multilayered biodegradable drug delivery device of claim 1, wherein the targeted site is a site of a vascular condition selected from the group consisting of vascular injury, vascular inflammation, ischemia-reperfusion injury, endotoxin-induced vascular damage, vascular trauma, graft failure, and thrombosis.

14. A method of fabricating a multilayered biodegradable drug delivery device, the method comprising:
providing a first layer comprising about 70% to about 95% of PLGA and about 5% to about 30% of gelatin, a second layer comprising 25% to about 70% of PLGA and about 30% to about 75% of gelatin, and an intermediate layer comprising about 55% to about 85% of PLGA and about 15% to about 45% of gelatin;
positioning the intermediate layer between the first layer and the second layer;
loading a Resolvin D1 (RvD1) solution between the first layer and the intermediate layer and between the intermediate layer and the second layer, is in the range of about 1 ng to about 10 μg; and
forming the multilayered biodegradable drug delivery device.

15. The method of claim 14, wherein the RvD1 solution comprises a Resolvin D1 dissolved in a pharmaceutically acceptable solvent, wherein the pharmaceutically acceptable solvent comprises any one of ethanol, methanol, isopropanol, dimethyl sulfoxide, acetonitrile, polyethylene glycol, propylene glycol, glycerol, phosphate-buffered saline containing a solubilizing agent, an albumin-containing solution, a cyclodextrin-containing solution, a lipid-based carrier, or combinations thereof.

16. A method of treating a vascular condition using a multilayered biodegradable drug delivery device, the method comprising:
applying the multilayered biodegradable drug delivery device to a blood vessel of a subject at a targeted site, wherein the device comprises:
a first layer comprising about 85% PLGA and about 15% gelatin;
an intermediate layer comprising about 75% PLGA and about 25% gelatin;
a second layer comprising about 50% PLGA and about 50% gelatin; and
a specialized pro-resolving mediator (SPM) is loaded between the first layer and the intermediate layer, and between the intermediate layer and the second layer, wherein the SPM is a Resolvin D1 (RvD1) dissolved in ethanol to obtain an RvD1 solution; and
facilitating a localized release of the RvD1 at the targeted site.

17. The method of claim 16, wherein the subject is a human.

18. The method of claim 16, wherein the first layer is disposed toward an outer surface of the blood vessel and the second layer is disposed toward an inner surface of the blood vessel.

19. The method of claim 16, wherein the targeted site is a site of a vascular condition selected from the group consisting of vascular injury, vascular inflammation, ischemia-reperfusion injury, endotoxin-induced vascular damage, vascular trauma, graft failure, and thrombosis.

20. The method of claim 16, wherein the released RvD1 inhibits a nuclear factor-κB (NF-κB)κactivation in vascular smooth muscle cells, reduces vascular smooth muscle cell proliferation, and reduces vascular smooth muscle cell migration.
